(19) United States
(12) Patent Application Publication
(10) Pub. No.: US 2025/0109294 A1
(43) Pub. Date: Apr. 3, 2025

(54) FOOD-GRADE THERMALLY STABLE COLORANTS AND METHODS OF PRODUCING THE SAME

(71) Applicant: GAVAN TECHNOLOGIES LTD, Shlomi (IL)

(72) Inventors: Gennady ROSENBLAT, Haifa (IL); Itai COHEN, Givatayim (IL)

(73) Assignee: GAVAN TECHNOLOGIES LTD, Shlomi (IL)

(21) Appl. No.: 18/832,095

(22) PCT Filed: Jan. 24, 2023
(86) PCT No.: PCT/IL2023/050081

§ 371 Date: Jul. 22, 2024

Related U.S. Application Data
(60) Provisional application No. 63/267,071, filed on Jan. 24, 2022.

(51) Int. Cl. C09B 61/00; A23L 5/46; A61K 8/64; A61Q 19/00; C11D 3/40

(52) U.S. Cl. CPC C09B 61/00; A23L 5/46; A61K 8/64; A61Q 19/00; C11D 3/40; A61K 2800/43

(57) ABSTRACT

Colorants of a natural origin having improved physical-chemical properties, such as light and thermal stability, compositions and articles comprising and/or made of such colorants are provided.

### FIELD OF INVENTION

The present invention relates to the field of food colorants, specifically thermally stable phycobiliprotein based food colorants originating from spirulina, related products, and processes for the manufacture thereof.

### BACKGROUND OF THE INVENTION

Food colorants are the category of food additives that is used in the food industry. Among food colorants characterized by higher stability are compounds having synthetic origin. However, as the awareness to the safety and quality of food products rises, use of certain synthetic colorants becomes less popular, forcing the manufacturers to look for valid alternatives. One of the routes is replacing artificial dyes with natural pigments. Unfortunately, the applicability of natural colorants is often limited, mainly due to their sensitivity to pH, light, high temperature, and other factors.

Numerous attempts have been made to overcome the challenges of natural food colorants such as encapsulation, emulsification with lipid compounds, utilization of different types of stabilizer, etc., but the vast majority of those attempts had limited success.

C-phycocyanine is a natural blue molecule belonging to the phycobiliproteins group. C-phycocyanine is used as natural colorant mainly for dairy products and yogurts (Moreira et al. 2012; Dewi et al. 2018; Kaur et al. 2019; Mohammadi-Gouraji et al. 2019; Campos Assumpcao de Amarante et al. 2020). Nevertheless, use of C-phycocyanine in food and beverages is still limited due to its sensitivity to heat, light, and acidic conditions.

Given the above, substituting synthetic colorants by natural and healthy products is one of the major challenges of the food industry. Improving physical-chemical characteristics of natural colorants, thus providing an alternative to the synthetic ones, remains a long and unmet need.

### SUMMARY OF THE INVENTION

It is a principal object of the invention to provide food colorants of a natural origin having improved physical-chemical properties, such as light and thermal stability.

According to some embodiments the invention provides stable phycocyanine-based colorant characterized by “L” coordinate in the range of 40.90 to 58.1 according to the Cie L*,a*,b* color coordinates.

According to some embodiments the invention provides a composition comprising a stable phycocyanine-based colorant characterized by “L” coordinate in the range of 40.90 to 58.1 according to the Cie L*,a*,b* color coordinates.

According to some embodiments the invention provides a stable phycocyanine-based colorant system in a form of a powder characterized by a particle size of less than 15.0 μm.

According to some embodiments the invention provides an article comprising a stable phycocyanine-based colorant characterized by “L” coordinate in the range of 40.90 to 58.1 according to the Hunter L.A.B color coordinates.

### BRIEF DESCRIPTION OF THE DRAWINGS

FIG. 1 is a graphic representation of Cie L*, a*, b* values of phycocyanine-based colorant obtained from dry spirulina;

FIG. 2 demonstrates Cie L*,a*,b* coordinates and calculated delta E value of microparticles obtained from differently treated phycocyanine aggregates; and,

FIG. 3 is a graphic representation of Cie L*, a*, b* values of microparticles obtained from differently treated phycocyanine aggregates.

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to some embodiments, the invention provides a stable phycocyanine-based colorant characterized by “L” coordinate in the range of 40.90 to 58.1 according to the Cie L * a * b scale. This scale is used for quantifying the shades of products. The scale principle is based on 3 axes. Axis, L * that quantifies the whiteness or darkness of the product, where 0 is dark “black” and 100 is light or “white”. In the axis, a * −red (100 (+ to green (100−)). On the b * axis is yellow (100+) to blue (100−). Each color − hue − can be quantified by determining the above three values. In addition to L * b * a * values, there are a number of indexes for characterizing specific colors and effects. The root of the differential sum of L * a * b * squared is “Delta E” (dE):

$$Δ E = \sqrt{{{{( Δ L \text{*)}}^{2} + ( Δ a \text{*)}}^{2} + ( Δ b \text{*)}}^{2}}$$

dE value expresses the total color difference of all color values in one number. It is accepted that a dE value above 1.5 to 2.0 is the limit for distinguishing by the human eye.

As used herein the term “stable” refers, without limitation, to thermal stability. In the context of the invention, the term “thermal stability” or “thermally stable” refers, without limitation, to the situation when the change in the sample color in course of the pasteurization process (standard industrial pasteurization processes such as 85° C. for 60 seconds or 90° C. for 30 seconds) is almost not perceptible by the human eye, and Total Color Difference (delta E) between heat treated and non-treated samples is not higher than 2.7.

According to some embodiments of the above colorants, the colorant is further characterized by “a” coordinate in the range of (−3.5) to (−13.9) according to the Cie L * a * b color coordinates.

According to some embodiments of the above colorants, the colorant is further characterized by “b” coordinate in the range of (−12.48) to (−19) according to the Cie L * a * b color coordinates.

According to some embodiments of the above colorants, the colorant is a solid, a semi-solid or a liquid. As used herein, the term “semi-solid” refers, without limitation to a state that is in between a solid and a liquid. Another name for a semi-solid is a quasi-solid. At the microscopic scale, it has a disordered structure unlike the more common solids.

According to some embodiments, the above colorant is in the form of microparticle mass having an average particle size in the range of 0.3 μm to 4.1 μm.

According to some embodiments, the above colorant in the form of microparticle mass having an average particle size of 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, and 4.1 μm.

According to some embodiments, the invention provides a composition comprising the colorant according to the above embodiments.

According to some embodiments, the above composition is, without limitation, a suspension, homogenous suspension, a dispersion, and an emulsion.

According to some embodiments, the invention provides stable phycocyanine-based colorant in a form of a powder characterized by a particle size of less than 15.0 μm.

According to some embodiments, the above stable phycocyanine-based colorant, the colorant is further characterized by “a” coordinate in the range of (−3.5) to (−13.9) according to the Hunter L.A.B color coordinates.

According to some embodiments, the above stable phycocyanine-based colorant is further characterized by an average particle size in the range of 0.5 μm to 3.3 μm.

According to some embodiments, the above stable phycocyanine-based colorant is further characterized by an average particle size of 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, and, 3.3 μm.

According to some embodiments, the above stable phycocyanine-based colorant is further characterized by “b” coordinate in the range of (−12.48) to (−19) according to the Cie L*,a*,b* color coordinates.

According to some embodiments, the above stable phycocyanine-based colorant is a food-grade colorant. In the context of the invention, the term “food-grade” is meant to be understood as a material/substance/media that is safe for human consumption.

According to some embodiments, the above stable phycocyanine-based colorant is characterized by “L” coordinate in the range of 40.90 to 58.1 according to the Cie L*,a*,b* color coordinates.

According to some embodiments, the invention provides an article comprising the colorant according to one or more of the above embodiments.

According to some embodiments, the above-article is, without limitation, a food article, an article used in cosmetics, such as, without limitation body-care product, facial-care product, hair-care product, and/or a baby-care; product, and/or an article used in chemical industry, such as, without limitation, a laundry product, a cleaning product, a dish washing product, and/or any other type of product that can benefit from the colorants of the invention.

According to some embodiments, the above-article might be, without limitation, syrup, soft drink, cream, foam, jelly, bar, instant product, gummy, pudding, jam, a solid product, dough-based product, a dairy product, dry soup mixture, fruit preparation, ripple, sweets and confectionary, beverages including hot beverages, alcoholic beverages, ice cream, sorbet, dairy alternatives, and chocolate.

According to some embodiments, the invention provides a process for the manufacture/extract of one or more of the above colorants comprising the steps of:
a. Extracting Spirulina in a Ca salt solution (0.5-5%) at 25-45 degrees Celsius for 1-10 hours;
b. Centrifugation of said solution at 5,000-10,000 RPM;
c. Ultrafiltration at 100 kDa, 50 kDa and 10 kDa using a series of PES filtration cassettes;
d. Flash-thermal treatment of the filtrate at 70-95 degrees Celsius;
e. Centrifugation of the filtrate at 5,000-10,000 RPM.

According to some embodiments of the above process, the yield is at least 80% and the purity is at least 1.0.

According to some embodiments of the above process, the Ca salt can be, without limitation, lactate, gluconate, acetate, carbonate, chloride or citrate.

According to some embodiments, the colorant according to the above embodiments of the invention is produced from spirulina biomass.

According to some embodiments, the colorant according to the above embodiments of the invention is produced from raw Spirulina and/or Spirulina-containing material.

According to some embodiments, the above colorant is a food colorant. In the context of the invention the term “food colorant” is meant to be understood as any color additive, dye, pigment, or substance that imparts color when it is added to food or drink. The food colorant may come in many forms including, but not limited to liquids, powders, gels, and pastes. The food colorants of the invention can be used in both commercial food production and domestic cooking.

According to some embodiments, the invention provides an edible media comprising the above colorants. As used herein, the term “edible” is meant to be understood, without limitation as safe and suitable for eating/consumption.

According to some embodiments, the invention provides food article prepared from the above edible media.

According to some embodiments, the invention provides food article comprising from the above edible media.

According to some embodiments, the above colorants, compositions, articles are vegetarian product and/or vegan. As used herein, the term “vegan” refers, without limitation, to a product containing no animal ingredients or animal-derived ingredients. As used herein, the term “vegetarian product” refers, without limitation, to a product that meets vegetarian standards by not including meat and animal tissue products.

The above colorants, compositions, articles according to one or more of the above embodiments, each individually or all together might be used, without limitation, in the artificial meat industry, artificial fish industry, printed food products, or any other type of products and/or industries and/or enterprises that may benefit from the present invention and its embodiments.

According to some embodiments, the invention provides a process for the manufacture of a colorant comprising the steps of:
a. Preparation of highly concentrated phycobiliprotein extract from raw Spirulina and/or Spirulina-containing raw material
b. Denaturation by thermal treatment to produce protein aggregates;
c. Isolation of protein aggregates;
d. Lyophilization; and,
e. Grinding of lyophilized phycobiliprotein-based aggregates.

According to some embodiments, Spirulina-containing raw material might be, without limitation, Spirulina encapsulated with starch, starch derivatives, alginate, proteins, gums, lipids, or any combination thereof. Preparation of highly concentrated phycobiliprotein extract may be carried out by direct extraction of raw material, or by using different methods for augmentation of phycobiliprotein concentration in low concentrated solution (including but not limited ultrafiltration, chromatography, low solvent volume for extraction etc.)

According to some embodiments, aggregation of phycobiliproteins in extracts can be done by any thermal treatment in the presence Ca⁺² ions, or any chemical, physical-chemical or physical methods causing phycobiliprotein aggregation, such as, without limitation, micro-wave etc.).

According to some embodiments, the separation of denatured phycobiliproteins-based aggregates, and their complexes and/or mixtures with other concomitant proteins from water-soluble compounds in extract is performed by filtration or centrifugation.

### EXAMPLES

### Example 1: Comparative Cie L*,a*,b* Coordinates of Phycocyanine-Based Microparticles Prepared from the Differently Extracted Phycocyanine

Phycocyanine was extracted in presence of CaCl₂) solution by four consequential extractions of the frozen spirulina encapsulated in alginate microbeads. Phycocyanine extracts from each extraction stage were used separately in further applications. After thermal treatment of the extracts at temperature between 85° C.-95° C., the protein aggregates were isolated, lyophilized, and grinded. Particle size distribution was studied by laser diffraction using Malvern Master Sizer. Obtained particles ranged between 0.357 μm and 4.06 μm with the median particle size 1.24 μm. The colorant powders obtained from the phycocyanine extracts of the first and the fourth extractions stage were characterized by Cie L*, a*, b* coordinates (Table 1)

TABLE 1
Comparative Cie L*, a*, b* value and color difference
between phycocyanine-based microparticles obtained
from the material of extraction 1 and 4.

Powder

| Treatment | Extraction | L* | A* | B* | dE |
|---|---|---|---|---|---|
| Untreated | Ex1 | 49 | −12 | −14 | 5.4 |
| Untreated | Ex4 | 48 | −9 | −19 | |

The data demonstrates the different shades of blue light of microbeads prepared from the differently extracted phycocyanine.

### Example 2: Cie L*,a*,b* Value and Graphic Representation of Phycocyanine-Based Microparticles Prepared from Dry Spirulina Extraction

Phycocyanine was extracted in presence of CaCl2) solution by extractions of dry spirulina. Phycocyanine extracts were separated from non-extracted material by centrifugation. After thermal treatment of the extract at temperature between 85° C.-95° C., the protein aggregates were isolated from the extract. The colorant obtained from the phycocyanine extract was characterized by Cie L*,a*,b* coordinates (Table 2).

| | Cie coordinates | | |
|---|---|---|---|
| Material | L* | a* | b* |
| Dry spirulina extract 1 | 28.30 | −13.46 | −6.53 |
| Dry spirulina extract 2 | 25.51 | −11.59 | −9.81 |

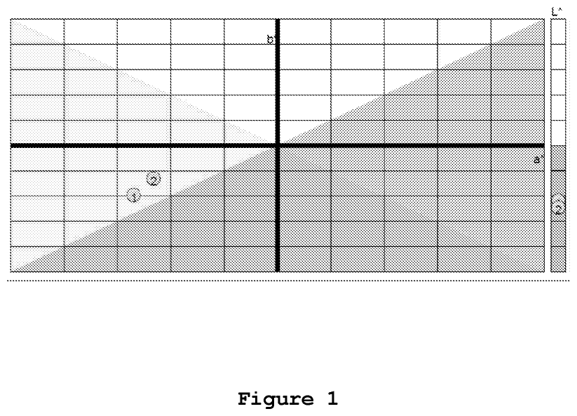

Graphic representation of Cie L*,a*,b* coordinates are shown in FIG. 1 which demonstrates different shades of blue light of microbeads prepared from the differently extracted phycocyanine. Phycocyanine extracts were obtained in the process of consequent four-stage phycocyanine extraction in presence of CaCl₂). The only difference was that one extract was obtained from the first extraction stage although another extract- from the fourth (final) extraction stage. A visual difference in blue shades of the samples was confirmed by difference in Cie L*,a*,b* coordinates. Calculated color difference (delta E value) between two samples was 5.4. This value is two time higher than a minimal delta E value−2.7, which is correspond to already non-visible differences between two colors.

### Example 3. Color Evaluation of Four Groups of Phycocyanine-Based Microparticles Obtained from Differently Treated Phycocyanine Aggregates

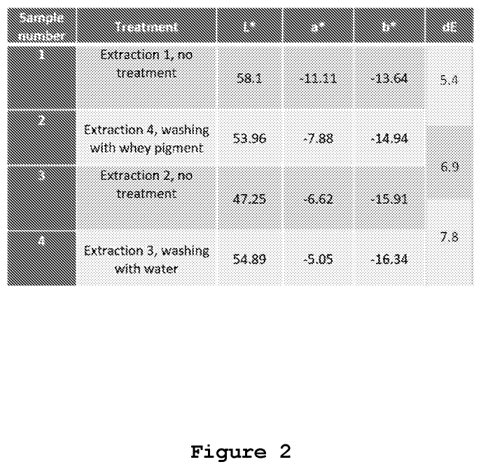

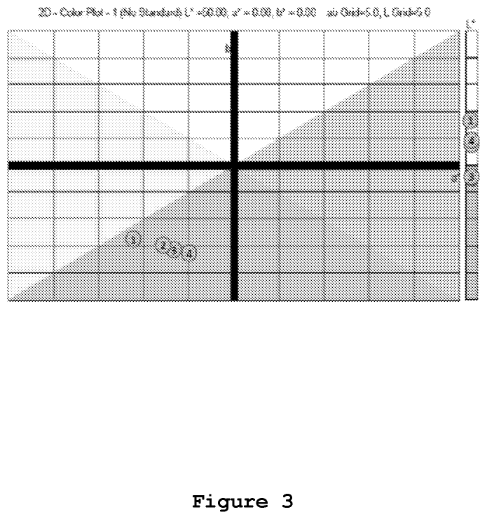

Phycocyanine was extracted from frozen spirulina by four consequential extractions in presence of CaCl₂. Phycocyanine extracts from each extraction stage were used separately in further applications. After thermal treatment of the extracts, the protein aggregates were separated from the liquid phase by filtration. After filtration, the protein aggregates from the third extraction were washed with water, although the protein aggregates from the fourth extraction were washed with the greenish-yellow filtrate obtained in process of fourth extract filtration. There was no washing for the protein aggregates from the first and second extraction. Obtained by those ways, four differently treated phycocyanine-based aggregates were lyophilized, grinded and characterized by Cie L*,a*,b* color coordinates, and color differences (dE) between three pair of differently prepared sample (sample 1 vs sample 2; sample 2 vs. sample 3; sample 3 vs. sample 4) were calculated. The results is shown in FIG. 2 and FIG. 3. FIG. 2 demonstrates the color evaluation of four group of phycocyanine-based microparticles which were prepared from the differently treated phycocyanine-based protein aggregates; the microbeads with four different shades of blue light were obtained. The data in FIGS. 2 and 3 demonstrate the different shades of blue light of microbeads prepared from the differently treated phycocyanine aggregates.

### Example 4. A Protocol for Extracting Phycocyanine (PC) from Spirulina

The protocol includes the following steps:
1. Crude extraction of Spirulina in a Ca salt solution (0.5-5%) at 25-45 C for 1-10 hours. The Ca salt being one of but not limited to Lactate, gluconate, acetate, carbonate, chloride or citrate.
2. Centrifugation of the solution at 5,000-10,000 RPM.
3. Gradual ultrafiltration at 100 kDa, 50 kDa and 10 kDa using a series of PES filtration cassettes.
4. A single step flash-thermal treatment of the filtrate at 70-95 C, causing denaturation around Calcium granules and precipitation.
5. Centrifugation of the filtrate at 5,000-10,000 RPM.

This results in 80% recovery of phycocyanine with average purity of 1.0-1.5 (A₆₂₀/A₂₈₀). SDS PAGE analysis, showed the presence of two subunits α (16 kD) and β (17 kD) in the purified phycocyanine.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on,” “attached” to, “operatively coupled” to, “operatively linked” to, “operatively engaged” with, “connected” to, “coupled” with, “contacting,” etc., another element, it can be directly on, attached to, connected to, operatively coupled to, operatively engaged with, coupled with and/or contacting the other element or intervening elements can also be present. In contrast, when an element is referred to as being “directly contacting” another element, there are no intervening elements present.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

All publications, patent applications, patents, and other references mentioned in the disclosures of these publications in their entireties are hereby incorporated by reference into this application in order to more fully describe the state of the art to which this invention pertains. In case of conflict, the patent specification, including definitions, will prevail. In addition, the materials, methods, and examples are illustrative only and not intended to be limiting. Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

1.-25. (canceled)

26. A stable phycocyanine-based colorant characterized by “L” coordinate in the range of 25.51 to 58.1 according to the Cie L*,a*,b* color coordinates.

27. The colorant of claim 26, further characterized by “a” coordinate in the range of (−3.5) to (−13.9); and/or “b” coordinate in the range of (−6.53) to (−19) according to the Cie L * a * b color coordinates.

28. The colorant of claim 26, wherein the colorant is a solid, a semi-solid, or a liquid.

29. The colorant of claim 26, in the form of microparticle mass having an average particle size in the range of 0.3 μm to 4.1 μm.

30. The colorant of claim 26, wherein the colorant is thermally stable.

31. A composition comprising the colorant of claim 26.

32. The composition of claim 31, selected from the group consisting of suspension, homogenous suspension, dispersion, and emulsion.

33. An article comprising the colorant of claim 26.

34. The article of claim 33, wherein the article is a food article.

35. The article of claim 34 selected from the group consisting of syrup, soft drink, hot beverage, jelly, bar, instant product, pudding, jam, a solid product, dough-based product, and a dairy product.

36. The article of claim 33, wherein the article is an article used in chemical industry.

37. The article of claim 36 selected from the group consisting of a laundry product, a cleaning product, and a dish washing product.

38. The article of claim 33, wherein the article is an article used in cosmetics.

39. The article of claim 38 selected from the group consisting of a body-care product, facial-care product, hair-care product, and a baby-care product.

40. A stable phycocyanin-based colorant in a form of a powder characterized by a particle size of less than 15.0 μm.

41. The colorant of claim 40, characterized by an average particle size in the range of 0.5 μm to 3.3 μm.

42. The colorant of claim 40, further characterized by “L” coordinate in the range of 40.90 to 58.1 according to the Cie L*,a*,b* color coordinates.

43. The colorant of claim 40, further characterized by “a” coordinate in the range of (−3.5)-(−13.9) according to the Cie L*,a*,b* color coordinates.

44. The colorant of claim 40, further characterized by “b” coordinate in the range of (−12.48) to (−19) according to the Cie L*,a*,b* color coordinates.

45. The colorant of claim 40, wherein the colorant is a food grade colorant.
